(19) United States
(12) Patent Application Publication
(10) Pub. No.: US 2006/0110779 A1
(43) Pub. Date: May 25, 2006

(54) Method for detecting a lectin-like substance

(75) Inventors: Huan-Yao Lei, Tainan City (TW); Chih-Peng Chang, Yuanlin Township (TW)

Correspondence Address: LADAS & PARRY, 26 WEST 61ST STREET, NEW YORK, NY, 10023, US

(73) Assignee: NATIONAL CHENG KUNG UNIVERSITY

(21) Appl. No.: 11/284,009

(22) Filed: Nov. 21, 2005

(30) Foreign Application Priority Data
Nov. 22, 2004 (TW) 093135900

(51) Int. Cl. G01N 33/574; A61K 38/16

(52) U.S. Cl. 435/7.23; 514/8

(57) ABSTRACT

The present invention provides a method for detecting lectin-like substances, which comprises the steps of (a) providing a first carbohydrate molecule and a test target, wherein the first carbohydrate molecule specifically binds to the lectin-like substance to be detected; (b) contacting the test target with the first carbohydrate molecule so that the lectin-like substance to be detected in the test target, if any, forms a complex with the first carbohydrate molecule; (c) removing the test target which does not form a complex with the first carbohydrate; (d) providing a detection unit comprising a second carbohydrate molecule and a reporter linked to the second carbohydrate molecule, wherein the second carbohydrate molecule is capable of specifically binding to the lectin-like substance to be detected; (e) contacting the detection unit of Step (d) with the complex formed in Step (b); (f) removing the detection unit which does not bind to the complex formed in Step (b); and (g) detecting the presence or absence of the reporter wherein the presence of the reporter indicates the presence of the lectin-like substance to be detected in the test target.

### BACKGROUND OF THE INVENTION

1. Field of the invention

The invention relates to a method for detecting lectin-like substances.

2. Description of the Related Art

Lectin is a glycoprotein capable of binding to certain monosaccharide molecules, which can agglutinate cells and bind to specific carbohydrates or carbohydrate-containing compounds. Lectin widely exists in nature. Lectin and its analogues can be found in plants, microorganisms and animals. In particular, plant seeds are rich in lectin. Lectin is considered a substance guarding plants from environmental toxins.

Lectin was originally named because of its ability to agglutinate red blood cells. Due to its carbohydrate-binding moiety, lectin has the ability to specifically bind to carbohydrate molecules, such as mannose, glucose, N-acetyl glucosamine, and galactose. Therefore, it is usually used in researches concerning glycoproteins on cell surfaces. In immunology, lectin is a potent mitogenic factor for stimulating lymphocyte proliferation. Furthermore, because the degree of glycosylation influences the malignancy of cells and metastatic activities in the development of cancerous cells, lectin has been used in alternative tumor therapies. As a result, detecting lectin having various functions from natural sources is important for new drug and health food developments.

Lectin is conventionally purified and detected with affinity chromatography, wherein its activity in agglutinating red blood cells is monitored. However, such procedure is time consuming and laborious. It cannot rapidly detect lectin and cannot be used to test on multiple samples at the same time.

### SUMMARY OF THE INVENTION

The invention provides a method for rapidly detecting lectin-like substances, which can detect multiple samples at the same time.

One object of the invention is to provide a method for detecting a lectin-like substance, which comprises the steps of:
(a) providing a first carbohydrate molecule and a test target, wherein the first carbohydrate molecule specifically binds to the lectin-like substance to be detected;
(b) contacting the test target with the first carbohydrate molecule so that the lectin-like substance to be detected in the test target, if any, forms a complex with the first carbohydrate molecule;
(c) removing the test target which does not form a complex with the first carbohydrate;
(d) providing a detection unit comprising a second carbohydrate molecule and a reporter linked to the second carbohydrate molecule, wherein the second carbohydrate molecule is capable of specifically binding to the lectin-like substance to be detected;
(e) contacting the detection unit of Step (d) with the complex formed in Step (b);
(f) removing the detection unit which does not bind to the complex formed in Step (b); and
(g) detecting the presence or absence of the reporter wherein the presence of the reporter indicates the presence of the lectin-like substance to be detected in the test target.

Preferably, the lectin-like substance is a lectin-like substance with anticancer activity, and more preferably, a lectin-like substance with anti-hepatoma activity.

### BRIEF DESCRIPTION OF THE DRAWINGS

FIG. 1. Cellular ELISA to detect lectins and lectin-like substances. ML-1 cells (1.5×10⁴) in a 96-well plate were fixed by 3.7% formaldehyde and incubated with various concentrations of Con A (a), WGA (b), RCA-1 (c), water extract from mung bean (d), or tropical violet, ivy gourd or night-fragrant flower (e) for 1 h at 37° C. Different biotinylated carbohydrate-PAA complexes (25 μg/mL) were used to detect the bound lectins and lectin-like substances. The peroxidase-sreptavidin conjugate was added and TMB substrate color was detected with a spectrophotometer at a wavelength of 450 nm.

FIG. 2. Carbohydrate-PAA-based ELISA to detect lectins and lectin-like substances. Different carbohydrate-PAA was coated on a 96-well plate and incubated with various concentrations of Con A (a), WGA (b), RCA-1 (c), water extract from mung bean (d), or tropical violet, ivy gourd or night-fragrant flower (e). Different biotinylated carbohydrate-PAA complexes (10 μg/mL) were used to detect the bound lectins and lectin-like substances. The peroxidase-sreptavidin conjugate was added and TMB substrate color was detected with a spectrophotometer at a wavelength of 450 nm.

FIG. 3. Detection of lectins bound to ML-1 cell surface by carbohydrate-polyacrylamide-biotin complex. ML-1 cells (2×10⁵/mL) were incubated with different concentrations of Con A, RCA-1, and WGA at 4° C. for 1 h. The bound lectin of various doses was detected using 100 μg/mL carbohydrate-polyacrylamide (PAA)-biotin complex and streptavidin-FITC with FACSCalibur™. The binding specificity of the biotinylated carbohydrate-PAA complex to Con A, WGA, or RCA-1-bound ML-1 cells was verified with different biotinylated carbohydrate-PAA complexes. Methyl α-mannopyranoside, N-acetyl glucosamine, or galactose was added to competitively block the binding.

FIG. 4. Lectin bound to tumor cell lines and splenocytes. ML-1 (a), CT-26 (b), Huh-7 (c), and splenocytes (d) at 1×10⁵/mL were incubated with 1 μg/mL of lectin-conjugated fluorescein for 30 min at 37° C. The binding was detected with FACSCalibur™.

FIG. 5. Apoptosis induced by lectins. ML-1 (a), CT-26 (b), Huh-7 (c) at 1×10⁵/mL were co-cultured with Con A, WGA, RCA-1 or PHA-E at various concentrations for 24 h. The apoptosis was quantified using Annexin V with FACSCalibur™.

FIG. 6. Proliferation induced by lectins on mouse splenocytes. Murine splenocytes (2×10⁵/mL) were cultured with various concentrations of Con A, WGA, RCA-1 or PHA-E for 72 h. The proliferation was detected by H³-thymidine incorporation.

FIG. 7. Detection of mannose-binding lectin-like substance from Sauropus androgynus extracts. The proteins of Sauropus androgynus (J04 and J05) extracts were obtained after precipitation with 70% saturated ammonium sulfate. O: original, P: ammonium sulfate-precipitated. (a) The mannose-PAA was coated on a 96-well plate and incubated with various concentrations of Con A, or Sauropus androgynus extracts at 4° C. overnight. Biotinylated mannose-PAA complexes (10 μg/mL) were used to detect the bound lectin-like substances. The peroxidase-sreptavidin conjugate was added and TMB substrate color was detected with a spectrophotometer at a wavelength of 450 nm. (b) Jurkat T cells (2×10⁵/mL) were incubated with different concentrations of Con A, or Sauropus androgynus extracts at 4° C. for 1 h. The bound lectin-like substance of various doses was detected using 100 μg/mL mannose-PAA-biotin complex and streptavidin-FITC with FACSCalibur™.

### DETAILED DESCRIPTION OF THE INVENTION

The invention relates to a method for rapidly detecting a lectin-like substance that can be used to simultaneously test on multiple samples.

The method according to the invention for detecting a lectin-like substance comprises the steps of:
(a) providing a first carbohydrate molecule and a test target, wherein the first carbohydrate molecule specifically binds to the lectin-like substance to be detected;
(b) contacting the test target with the first carbohydrate molecule so that the lectin-like substance to be detected in the test target, if any, forms a complex with the first carbohydrate molecule;
(c) removing the test target which does not form a complex with the first carbohydrate;
(d) providing a detection unit comprising a second carbohydrate molecule and a reporter linked to the second carbohydrate molecule, wherein the second carbohydrate molecule is capable of specifically binding to the lectin-like substance to be detected;
(e) contacting the detection unit of Step (d) with the complex formed in Step (b);
(f) removing the detection unit which does not bind to the complex formed in Step (b); and
(g) detecting the presence or absence of the reporter wherein the presence of the reporter indicates the presence of the lectin-like substance to be detected in the test target.

As used herein, the term “a lectin-like substance” refers to a substance having the ability to bind to a specific carbohydrate. The lectin-like substance may be a micromolecule or a macromolecule, preferably a macromolecule; and more preferably a glycoprotein. Such substance is named because it has characteristics similar to lectin. The lectin-like substance to be detected according to the invention may be a known substance or a substance yet to be identified.

As used herein, the term “a test target” refers to a sample to be detected for the presence of a lectin-like substance. The target may be a material, mixture or extract derived from animals, plants or microorganisms, preferably a mixture or extract, and more preferably, a mixture or extract derived from plants.

As used herein, the term “a first carbohydrate molecule” or “a second carbohydrate molecule” refers to a molecule with a carbohydrate structure, which may be a carbohydrate molecule, a macromolecule comprising a carbohydrate moiety (such as a glycoprotein), a molecule with a carbohydrate structure attached to a carrier, or a carbohydrate molecule present on a cell surface. Preferably, the molecule with a carbohydrate structure is a carbohydrate molecule or a carbohydrate molecule present on a cell surface. More preferably, the molecule with a carbohydrate structure is a monosaccharide. The first and second carbohydrate molecules are capable of specifically binding to the lectin-like substance to be detected. The species of the first and second carbohydrate molecules are chosen in accordance with the object for detection. For instance, where detecting a lectin-like substance with anticancer activity is desired, carbohydrate molecules on the surface of tumor cells, cancerous cells or lymphocytes can be used. Carbohydrate molecules present on the surface of cells are well known to persons of ordinary skill in the art. See, for instance, Lalwani et al or Boland et al (Lalwani A K., Carey T E., Goldstein I J., Peters B P. Lectin binding characteristics of squamous cell carcinomas of the head and neck. Acta Oto-Laryngologica. 116(1):125-31, 1996; Boland C R., Martin M A., Goldstein I J. Lectin reactivities as intermediate biomarkers in premalignant colorectal epithelium. [Review] Journal of Cellular Biochemistry-Supplement. 16G:103-9, 1992).

In one embodiment of the invention, the first carbohydrate molecule in Step (a) is attached to the surface of a carrier. As used herein, the term “a carrier” refers to an inert support that does not react with the carbohydrate molecules or the test target, or interfere with the reaction therebetween. For example, the carrier can be made of resins. According to the invention, the first carbohydrate molecule is a carbohydrate molecule attached to the surface of a carrier. The carbohydrate molecule can also be present on the surface of a cell attached to a carrier. The method for attaching the first carbohydrate molecule to the surface of a carrier is well known to persons of ordinary skill in the art. For example, the coating method used in an enzyme-linked immunosorbent assay system can be utilized.

Based on the object for detection, different cells with a carbohydrate molecule present on the surface are chosen. For instance, when detecting a lectin-like substance with anticancer activity is desired, a tumor cell, cancer cell or lymphocyte is chosen. In one embodiment of the invention, the lectin-like substance with anticancer activity is a lectin-like substance with anti-hepatoma actitiy or an anti-colon cancer activity. Preferably, the lectin-like substance has an anti-hepatoma activity. In one embodiment of the invention, a hepatoma cell or a colon cancer cell is used in Step (a).

The first carbohydrate molecule and the test target can be contacted in Step (b) in a manner conventionally known in the art. The first carbohydrate molecule and the test target are contacted under an appropriate condition, such as suitable ion strength, pH value and temperature, such that the first carbohydrate molecule and the test target form a complex if the lectin-like substance to be detected is present in the test target.

The test target that does not form a complex with the first carbohydrate molecule in Step (b) is removed in Step (c). The removal can be performed in a manner conventionally known to persons of ordinary skill in the art. For example, the test target can be removed by removing the reaction solution or by washing.

The “detection unit” according to the invention comprises a second carbohydrate molecule and a reporter linked to the second carbohydrate molecule. As used herein, the term “a reporter” refers to a substance that emits a signal when the detection unit binds to the complex formed between the first carbohydrate molecule and the lectin-like substance to be detected. The reporter can be a molecule comprising a conventional dye, fluorescence, luminescence, enzyme, or reporter protein. Preferably, the reporter comprises a fluorescence moiety. The second carbohydrate molecule and the reporter can be linked in accordance with technology well known to persons of ordinary skill in the art. For example, enzymatic catalysis and chemical reaction can be used based on the species of and properties of the second carbohydrate molecule and the reporter.

According to the invention, if the first carbohydrate molecule and the second carbohydrate molecule have the same carbohydrate structure, the method of the invention can be used to detect the lectin-like substances capable of binding to the carbohydrate structure. If the first carbohydrate molecule and the second carbohydrate molecule have different carbohydrate structures, the method of the invention can be used to detect the lectin-like substances capable of binding to both carbohydrate structures. Preferably, the first carbohydrate molecule and the second carbohydrate molecule have the same molecule structures.

The detection unit and the complex formed between the test target and the first carbohydrate molecule can be contacted in a conventional manner. The detection unit and the complex are contacted under an appropriate condition, such as suitable ion strength, pH value and temperature, such that the detection unit binds to the complex. Preferably, the detection unit and the complex are contacted in the same manner and under the same condition used in Step (b).

The detection unit which does not bind to the complex of the test target and the first carbohydrate is removed in Step (f). The removal can be performed in a manner conventionally known to persons of ordinary skill in the art. For example, the detection unit can be removed by removing the reaction solution or by washing.

The reporter can be detected in accordance with conventional methods depending on the species of the signal of the reporter. For instance, visible light/UV spectrophotometer, fluorescence spectrophotometer, luminescence spectrophotometer and flow cytometry can be used for detecting the reporter.

In one embodiment of the invention, an enzyme-linked immunosorbent assay (ELISA) system is used for detecting the lectin-like substance. The ELISA system can advantageously detect multiple samples in one manipulation. In addition, the system is also convenient to manipulate, and its reagents and procedures have been established. If the carbohydrate molecule is present on a cell, a cell-based enzyme-linked immunosorbent assay system can be used.

In one embodiment of the invention, the lectin-like substance is a lectin-like substance with anticancer activity. More preferably, the lectin-like substance is a lectin-like substance with anti-hepatoma activity.

Because the occurrence of tumor and/or cancer is significantly related to the immune system, the method of the invention can be used to detect a lectin-like substance capable of stimulating lymphocyte proliferation. In one embodiment, lymphocytes are co-cultured with the test target and monitored with respect to their proliferation due to the modulation of the test target.

In another embodiment, the method of the invention is used to detect a lectin-like substance with activity related to tumor/cancer mechanisms, such as activity related to cancer cell apoptosis or cancer cell cytotoxicity stimulation.

The following Examples are given for the purpose of illustration only and are not intended to limit the scope of the present invention.

### Example 1

### Detection of Lectin-Like Substances (1)

Cell lines and lectins.

Hepatoma cell line, ML-1, derived from BALB/c mice was kindly provided by Dr. C. P. Hu (Department of Medical Research, Veterans General Hospital, Taipei, Taiwan, ROC).

Murine colon cancer cell line, CT-26, was obtained from the American Type Culture Collection.

Human hepatoma cell line, Huh-7, was kindly provided by Dr. M. D. Lai (Department of Biochemistry, Medical college of National Cheng Kung University, Taiwan, ROC).

All cell lines were cultured in DMEM (Gibco®, Grand Island, USA) supplemented with 10% FBS and penicillin-streptomycin. Lectins were purchased from Vector®, Burlingame, Calif., USA, and their characteristics are shown in Table 1.

TABLE 1

| Lectin | | |
|---|---|---|
| abbreviation | Species/source | Sugar specificity |
| Con A | Canavalia ensiformis | Glucose, |
| | (Jack bean) | Mannose |
| PSA | Pisum sativum | Glucose, |
| | (Pea) | Mannose |
| LCA | Lens culinaris | Glucose, |
| | (Lentil) | Mannose |
| WGA | Triticum vulgaris | N-acetyl glucosamine, |
| | (Wheat germ) | Sialic acid |
| RCA I | Ricinus communis | N-acetyl galactosamine, |
| | (Caster bean) | Galactose |
| GSL I | Griffonia simplicifolia | N-acetyl galactosamine, |
| | | Galactose |
| PHA-E | Phaseolus vulgaris | Complex structures |
| | (Red kidney bean) | |
| PHA-L | Phaseolus vulgaris | Complex structures |
| | (Red kidney bean) | |

Detection of cell-bound lectin or lectin-like substance by cell-based ELISA.

ML-1 (1.5×10⁴) cells were seeded on a 96-well plate for attachment overnight. The cells were then washed with DMEM once and fixed with 3.7% formaldehyde in PBS at room temperature for 15 minutes. After fixation, the cells were washed with PBS and incubated with lectins in staining buffer (DMEM containing 2% FBS and 0.1% NaN₃) at 37° C. for 1 h. After incubation, the cells were washed with PBS and then incubated with 25 μg/mL carbohydrate-polyacrylamide (PAA)-biotin complex in a binding buffer (PBS with 2% low-biotin FBS and 0.1% NaN₃) at room temperature for 1 h. After incubation and washing, 1 μg/mL streptavidin-conjugated peroxidase (Pharmignen®, San Diego, Calif.) was added to the wells at room temperature for another 30 minutes. After the final wash, the substrate teramethylbenzidine (TMB) was added for color formation and detected with a spectrophotometer at a wavelength of 450 nm.

Detection of lectin or lectin-like substance by carbohydrate-PAA-based ELISA.

Five μg/mL carbohydrate-PAA was coated on a non-treated 96-well plate at 4° C. overnight. After 3 washes with PBS, the plate was blocked with 1% BSA for 2 hr at room temperature. Then lectins or water-extract from Asystasia gangetica (tropical violet), Coccinia grandis (ivy gourd), or Telosma cordata (night-fragrant flower) were added to the wells at 4° C. overnight. After incubation, the plate was washed with PBS and then incubated with 10 μg/mL carbohydrate-PAA-biotin complex in the binding buffer at room temperature for 1 h. After incubation and washing, 1 μg/mL streptavidin-conjugated peroxidase was added to the wells at room temperature for another 30 minutes. After the final wash, the substrate teramethylbenzidine (TMB) was added for color formation and detected with a spectrophotometer at a wavelength of 450 nm.

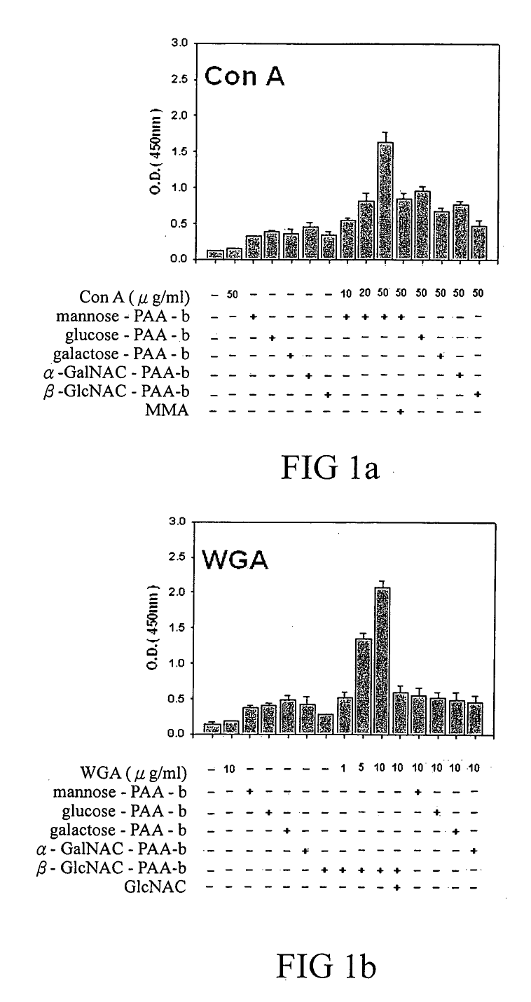

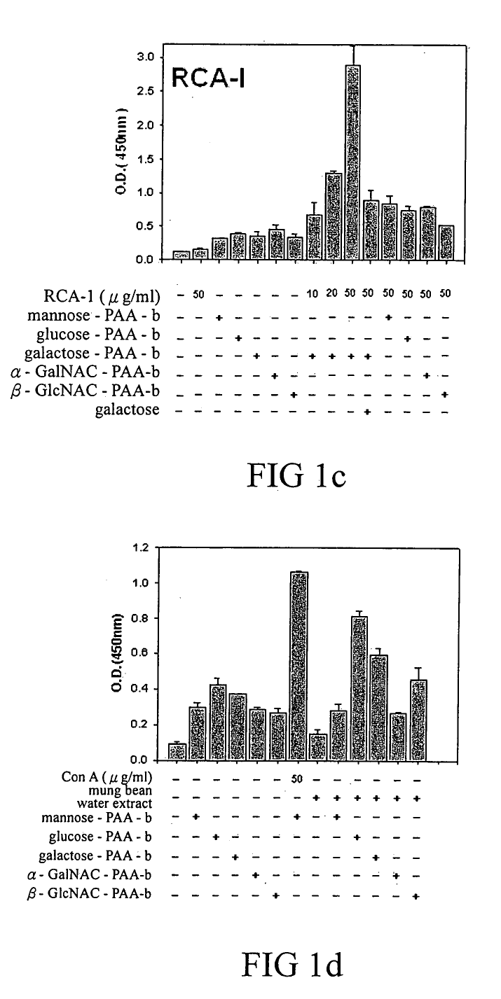

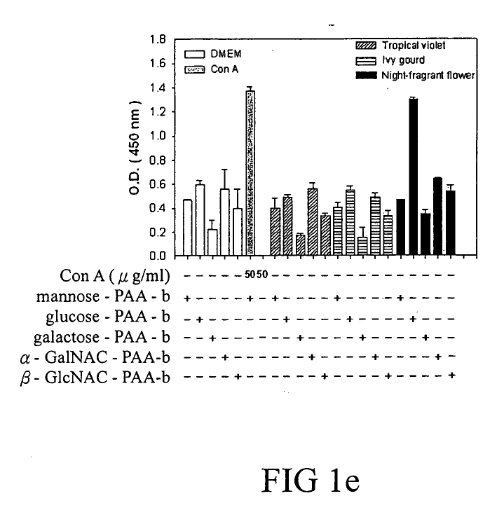

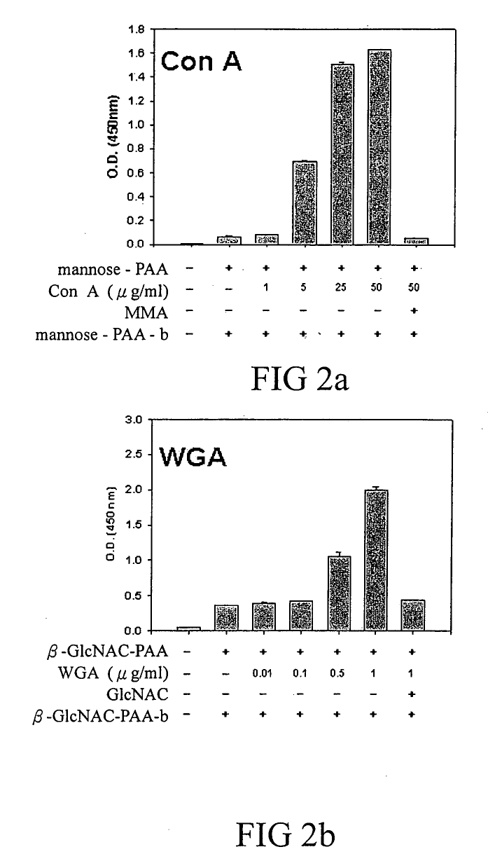

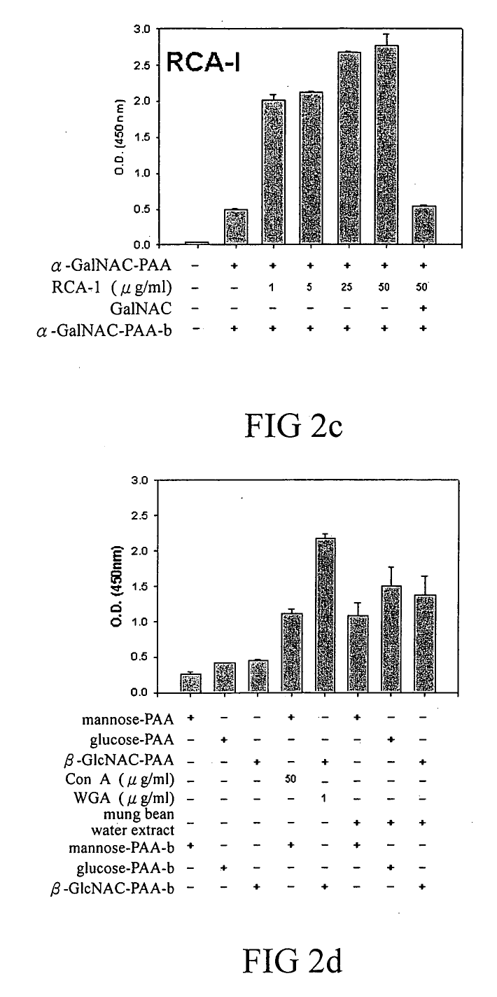

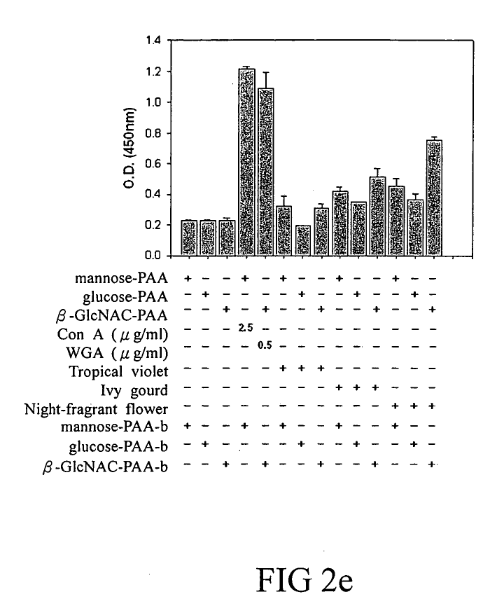

The results are shown in FIGS. 1 and 2.

In FIGS. 1a and 2a, Con A binding to ML-1 cells or mannose-PAA-coated well was detected specifically by mannose-PAA-biotin-complex, and when more Con A was added to cells or mannose-PAA-coated well, a higher optical density (O.D.) value of mannose-PAA-biotin-streptavidine-complex was observed, while glucose-PAA had only a low binding ability. Methyl α-mannopyranoside could competitively block this binding.

From FIGS. 1b and 2b, WGA binding to ML-1 cells or N-acetyl glucosamine-PAA-coated well was detected specifically by N-acetyl glucosamine-PAA-biotin-complex and N-acetyl glucosamine could competitively block this binding.

In FIGS. 1c and 2c, RCA-1 binding to ML-1 cells or N-acetyl galactosamine-PAA-coated well was detected specifically by galactose- or N-acetyl galactosamine-PAA-biotin-complex in a dose-dependent manner. The detection by galactose-PAA was greater than N-acetyl galactosamine-PAA. N-acetyl galactosamine could competitively block this binding.

Using the carbohydrate-specific binding ELISA, lectin-like substances in natural products were screened. The water extract from mungbean, tropical violet, ivy gourd or night-fragrant flower was added to ML-1 cells or carbohydrate-PAA-coated wells, then biotin-conjugated mannose-PAA, glucose-PAA, galactose-PAA, N-acetyl glucosamine-PAA, or N-acetyl galactosamine-PAA was used to detect the bound lectins.

As shown in FIGS. 1d and 2d, the mung bean water extract (V01160) contained the lectin-like substances that were glucose or galactose-specific, and detected by glucose-PAA or less by galactose-PAA. On the other hand, more lectin-like substances that were glucose or N-acetyl glucosamine-specific binding were detected in the Night-fragrant flower water extract, but less in the tropical violet or Ivy gourd water extract (FIGS. 1e and 2e).

### Example 2

### Detection of Lectin-Like Substances (2)

Detection of bound lectins on ML-1 cells by flow cytometry.

ML-1 (2×10⁵) cells after harvest by trypsinization were suspended in a staining buffer and then incubated with lectins at 4° C. for 1 h. After incubation, ML-1 cells were washed with PBS and centrifuged. The cells were then suspended in the binding buffer with 100 μg/mL carbohydrate-polyacrylamide (PAA)-biotin complex (GlycoTech®, Maryland, USA) at 4° C. for another 1 h. After incubation, ML-1 cells were washed with PBS and centrifuged. Finally, the cells were incubated with 1 μg/mL streptavidin-conjugated fluorescein (Serotec®, Oxford, UK) at 4° C. for 30 minutes and analyzed with flow cytometry at a wavelength of 488 nm.

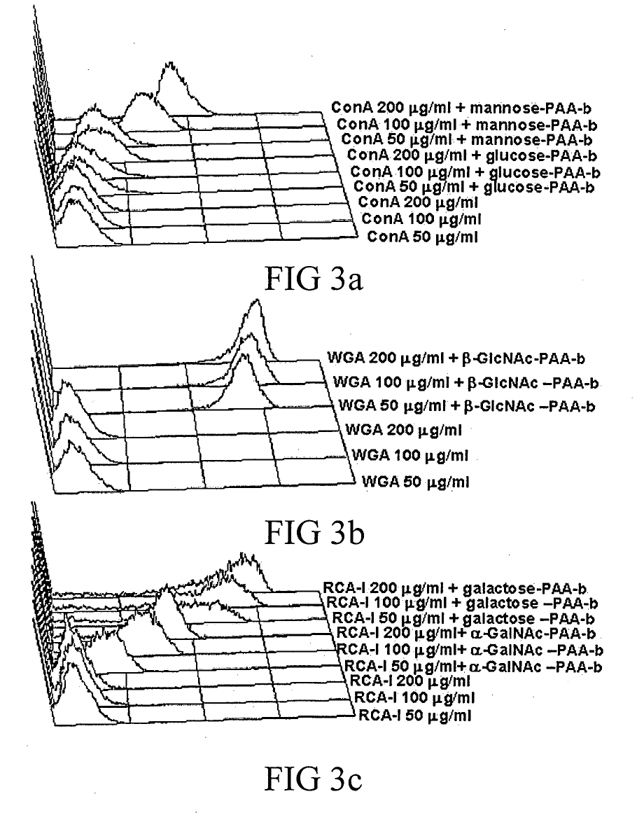

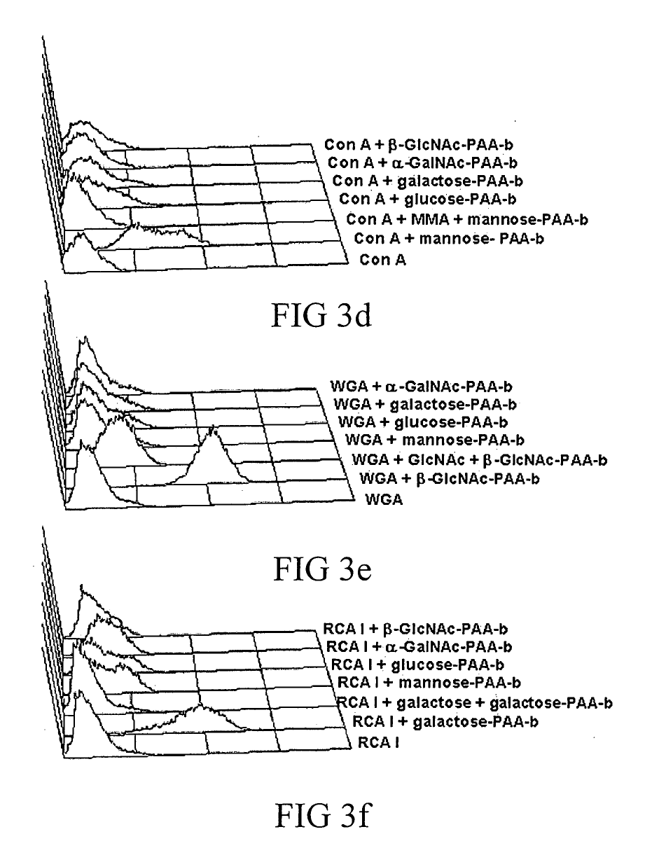

A cell-based binding assay was used. ML-1 cells were first incubated with Con A, RCA-1, or WGA for 1 h, while the lectin bound to carbohydrate residue on the cell membrane. Then biotin-conjugated carbohydrate-PAA was added to form a complex with the lectin, and the lectin-bound cells were detected by FITC-streptavidin. The results are shown in FIG. 3. The bond lectins on ML-1 cell could be specifically detected by carbohydrate-PAA-biotin complex with flow cytometry. The binding pattern is similar to the ELISA used in Example 1.

### Example 3

### Detection of Anti-Cancer Lectin-Like Substances

The lectin-like substances detected by ML-1 cell line in Examples 1 and 2 were subjected to further detections with other tumor cells and lymphocytes.

Lectin binding assay.

ML-1, CT-26, Huh-7 and murine splenocytes (1×10⁵) were suspended in a staining buffer and co-incubated with 5 μg/mL of fluorescein-conjugated lectin (Vector®, Burlingame, Calif.) for 30 minutes at 37° C. The binding capacity of lectin to cells was detected with flow cytometry.

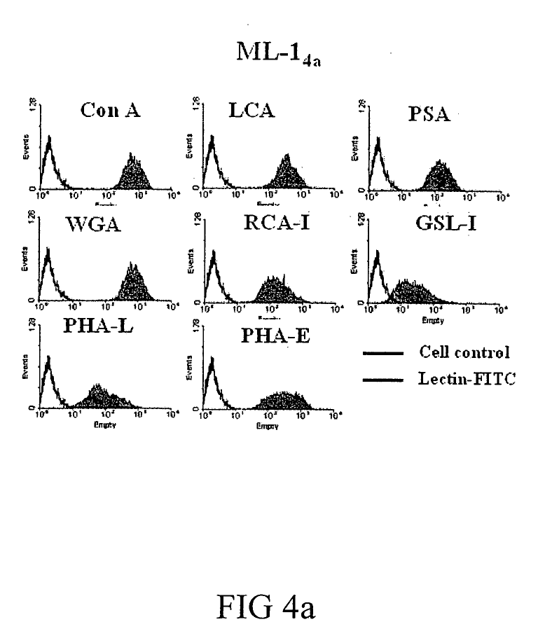

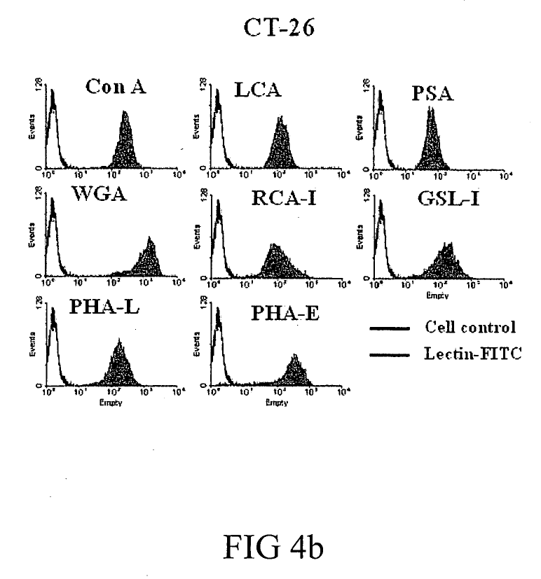

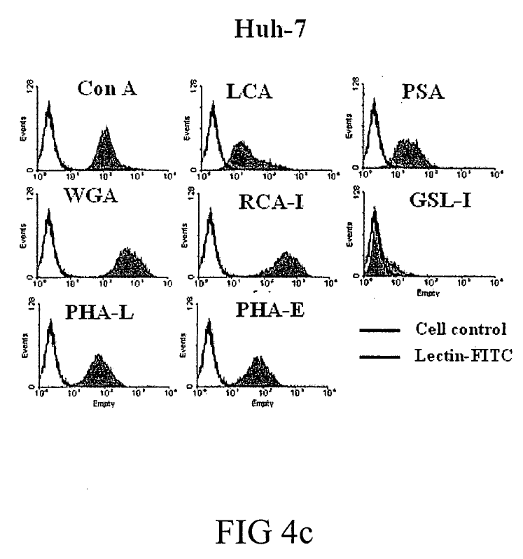

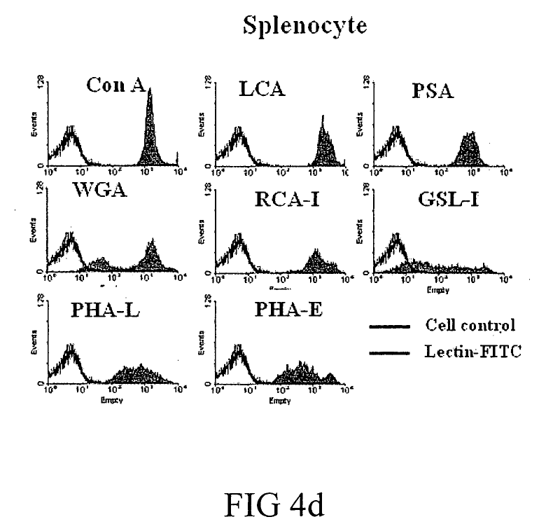

The results are shown in FIGS. 4a to 4d. Using FITC-lectin binding to murine hepatoma cell line ML-1, murine colon cancer cell line, or human hepatoma cell line Huh-7, the flow cytometric analysis showed that lectins such as Con A, LCA, PSA, WGA, RCA-1, GSL-1, PHA-L, and PHA-E can bind to these tumor cell lines with different affinity (FIGS. 4a, 4b, and 4c). The lectin binding intensity with different tumor cells varied slightly, indicating that the degree of glycosylation of the different tumor cells varied. The lectin binding to murine splenocytes was also tested. The fluorescent intensity detected on lymphocytes was greater than that on tumor cell lines (FIG. 4d), suggesting there were more carbohydrate moieties on lymphocytes.

Apoptosis assay.

ML-1, CT-26, and Huh-7 were harvested by trypsinization and seeded 1×10⁵ cells into each well of 12-well plates for 2 hours. Different concentrations of lectins were added into the plates that contained tumor cells. Cells were harvested 24 h later and the apoptosis was quantified by flow cytometry, using the Annexin™ V-PI kit (BioVision®, Mountain View, Calif.).

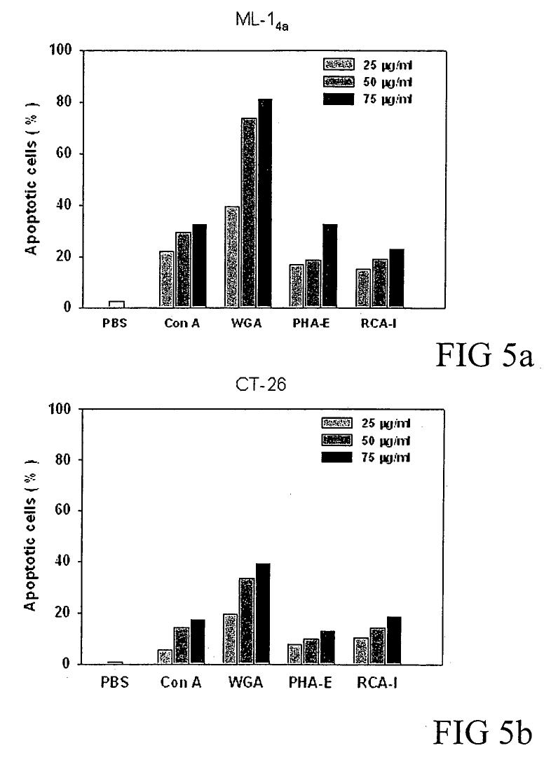

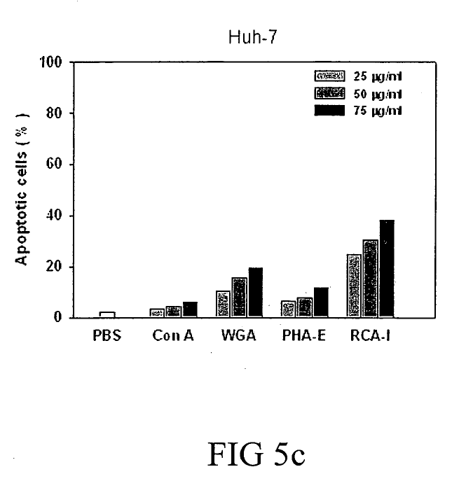

The response after lectin binding was studied further. As shown in FIG. 5, Con A, WOA, PHA-E, or RCA-1 could induce tumor cell (ML-1, CT-26, and Huh-7) apoptosis dose-dependently, although the sensitivity of various tumor cells was different, depending on the lectins used.

Mitogenic activities of lectins to splenocytes.

BABL/c mice (male, 8-10 weeks old) were purchased from National Laboratory Animal Center (Taipei, Taiwan), and maintained in the pathogen-free facility of the Animal Laboratory of National Cheng Kung University. The splenocytes were isolated from the spleen following a normal procedure. Then, 2×10⁵ lymphocytes were stimulated with various lectins in different concentrations for 72 h. Splenocyte proliferation was detected by H³-thymidine incorporation.

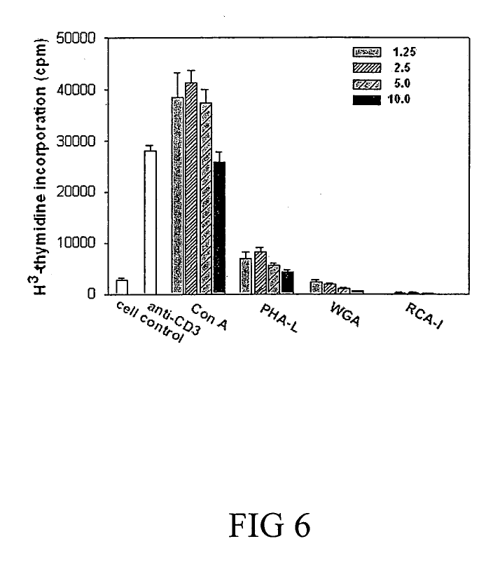

As shown in FIG. 6, Con A and PHA-E were mitogenic to murine splenocytes while WGA and RCA-1 were toxic to lymphocytes. The dose necessary for lymphocyte proliferation was lower than that for tumor cell apoptosis. Lymphocytes are thus more sensitive than tumor cells probably because of the greater carbohydrate moieties on lymphocytes.

### Example 4

### Detection of Mannose-Binding Lectin-like Substance from Vegetable Extracts

Vegetable extract.

Various species of vegetable were extracted: 20 g fresh edible portion and 40 mL distilled water were mixed to blend and then centrifuged at 10000 rpm for 10 min. The supernatant was collected and frozen for use.

Protein precipitation.

The proteins in Sauropus androgynus extract were precipitated with 70% saturated ammonium sulfate at 4° C. overnight, then centrifuged at 10000×g for 30 minutes. The precipitate was dissolved in water and subjected to dialysis for 24 h against 200 volumes of PBS.

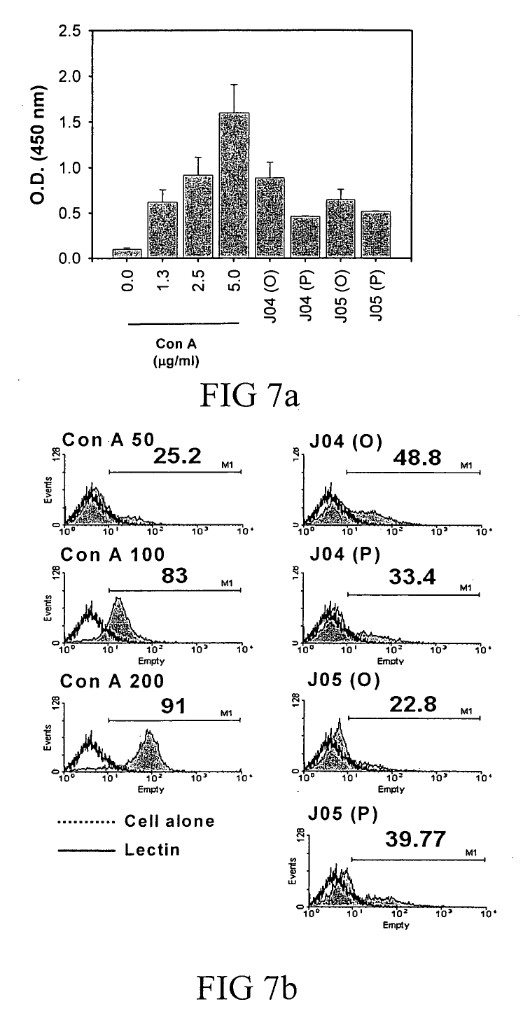

Using the carbohydrate-specific binding ELISA, lectin-like substances in natural vegetable extracts were screened and analyzed. In comparison with the carbohydrate-PAA binding ability of Con A, WGA or RCA-1, all lectin-like substances in natural vegetable were presented and summarized in Table 2. The extract from Sauropus androgynus contains higher lectin-like substances with mannose-specific binding. It was chosen for further study. The Sauropus androgynus extract were precipitated with 70% ammonium sulfate to collect the proteins, then mannose-specific binding ability was determined by mannose-PAA-biotin complex. As shown in FIG. 7a, both the original extract and the precipitated-proteins of the Sauropus androgynus extracts (J04 and J05, O: original extract, P: ammonium sulfate-precipitated extract) contained the mannose-binding ability as detected by mannose-PAA-biotin complex-based ELISA. Furthermore, these mannose-specific lectin-like substances can bind to Jurkat T cells as detected with flow cytometry (FIG. 7b).

TABLE 2

| | Con A | WGA | RCA-I |
|---|---|---|---|
| | (μg/mL) | (μg/mL) | (μg/mL) |
| Sample name | equivalent | equivalent | equivalent |
| Sauropus androgynus-2 (J05) | 2.942 | 0.832 | 0.65 |
| Toona sinensis | 2.748 | 0.776 | 0.167 |
| Sausopus androgynus-1 (J04) | 2.611 | 0.735 | 0.384 |
| Abelmoschus esculentus | 2.406 | 0.675 | 0.2 |
| Adansonia digitata | 2.282 | 0.639 | 0.784 |
| Moringa oleifera | 1.931 | 0.536 | 0.888 |
| Sesbania grandiflora | 1.556 | 0.427 | 0.519 |
| Telosma cordata | 0.602 | 0.147 | 0.465 |
| Coccinia grandis | 0.545 | 0.130 | 0.368 |
| Corchorus olitorius | 0.254 | 0.045 | 0.344 |

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A method for detecting lectin-like substances, which comprises the steps of:
(a) providing a first carbohydrate molecule and a test target, wherein the first carbohydrate molecule specifically binds to the lectin-like substance to be detected;
(b) contacting the test target with the first carbohydrate molecule so that the lectin-like substance to be detected in the test target, if any, forms a complex with the first carbohydrate molecule;
(c) removing the test target which does not form a complex with the first carbohydrate;
(d) providing a detection unit comprising a second carbohydrate molecule and a reporter linked to the second carbohydrate molecule, wherein the second carbohydrate molecule is capable of specifically binding to the lectin-like substance to be detected;
(e) contacting the detection unit of Step (d) with the complex formed in Step (b);
(f) removing the detection unit which does not bind to the complex formed in Step (b); and
(g) detecting the presence or absence of the reporter wherein the presence of the reporter indicates the presence of the lectin-like substance to be detected in the test target.

2. The method according to claim 1, wherein the first carbohydrate molecule is a monosaccharide.

3. The method according to claim 1, wherein the first carbohydrate molecule is present on the surface of a carrier.

4. The method according to claim 1, wherein the first carbohydrate molecule is present on the surface of a cell.

5. The method according to claim 4, wherein the cell is attached to the surface of a carrier.

6. The method according to claim 4, wherein the cell is a tumor cell, a cancer cell or a lymphocyte.

7. The method according to claim 1, wherein the test target is a mixture or an extract derived from animals, plants or microorganisms.

8. The method according to claim 1, wherein the second carbohydrate molecule is a monosaccharide.

9. The method according to claim 1, wherein the first carbohydrate molecule and the second carbohydrate molecule have the same carbohydrate structure.

10. The method according to claim 1, wherein the second carbohydrate molecule is present on the surface of a cell.

11. The method according to claim 10, wherein the cell is a tumor cell, a cancer cell or a lymphocyte.

12. The method according to claim 1, wherein the reporter comprises a fluorescence moiety.

13. The method according to claim 1, which is carried out with an enzyme-linked immunosorbent assay system.

14. The method according to claim 1, wherein the lectin-like substance is a lectin-like substance with anticancer activity.

15. The method according to claim 14, wherein the lectin-like substance with anticancer activity is a lectin-like substance with anti-hepatoma activity.

16. The method according to claim 14, wherein the lectin-like substance with anti-cancer activity is capable of stimulating lymphocyte proliferation.

17. The method according to claim 14, wherein the lectin-like substances with anti-cancer activity is capable of stimulating cancer cell apoptosis.
